(19) United States
(12) Patent Application Publication
(10) Pub. No.: US 2002/0044934 A1
(43) Pub. Date: Apr. 18, 2002

(54) THERAPIES FOR SMOKE AND/OR BURN INDUCED LUNG INJURY

(76) Inventors: FRANK C. SCHMALSTIEG, GALVESTON, TX (US); DANIEL L. TRABER, GALVESTON, TX (US)

Correspondence Address: CHRISTOPHER A HARE, ABGENIX INC, 7601 DUMBARTON CIRCLE, FREMONT, CA, 94555

(21) Appl. No.: 09/186,726

(22) Filed: Nov. 4, 1998

(51) Int. Cl. A61K 39/395

(52) U.S. Cl. 424/145.1

(57) ABSTRACT

In accordance with the present invention there are provided therapies for lung injuries following smoke inhalation and thermal injuries. In particular, the invention relates to the use of antibodies, and more particularly human antibodies, as therapeutics of such injuries. In preferred aspects of the invention, antibodies that bind to interleukin-8 (IL-8) and/or L-selectin, alone, or in combination with other antibodies that bind to other pro-inflammatory, inflammatory, and anti-inflammatory moieties that are induced in lung and burn lesions. In particularly preferred embodiments of the invention, human antibodies that bind to IL-8 are utilized and are demonstrated herein to possess therapeutic efficacy in in vivo models of the pro-inflammatory, inflammatory, and anti-inflammatory moieties that are induced in lung and burn lesions.

### BACKGROUND OF THE INVENTION

1. Summary of the Invention

In accordance with the present invention there are provided therapies for lung injuries following smoke inhalation and thermal injuries. In particular, the invention relates to the use of antibodies, and more particularly human antibodies, as therapeutics of such injuries. In preferred aspects of the invention, antibodies that bind to interleukin-8 (IL-8) and/or L-selectin, alone, or in combination with other antibodies that bind to other pro-inflammatory, inflammatory, and anti-inflammatory moieties that are induced in lung and burn lesions. In particularly preferred embodiments of the invention, human antibodies that bind to IL-8 are utilized and are demonstrated herein to possess therapeutic efficacy in in vivo models of the pro-inflammatory, inflammatory, and anti-inflammatory moieties that are induced in lung and burn lesions.

2. Background of the Technology

A common situation encountered in human trauma is a combination of lung injury secondary to both smoke inhalation and burns. Lung injury from smoke inhalation alone is generally successfully managed if proper support is given to the patient (1). However, lung injury associated with combined smoke inhalation and thermal burns is far more lethal than either injury alone (1). Both burn edema and lung oxidative damage are synergistically increased by the combined injury (2). A number of mechanisms may contribute to the increased injury. It is known that burns may result in acute constriction of mesenteric arteries with injury of the gut mucosa and increased bacterial translocation (3). In addition, reperfusion injury with activation of neutrophils is highly likely. These activated neutrophils would be expected to contribute to an increase in lung injury. Predictably, depletion of neutrophils before injury greatly reduces the pulmonary pathology. Consistent with this hypothesis, lipid peroxidation products in the lung and peripheral tissues are both increased over smoke or burn injury alone (35). In addition to these mechanisms, a large number of inflammatory cytokines are produced in both the lung and burn injury (6-8). A mixture of pro-inflammatory, inflammatory, and anti-inflammatory cytokines are produced in this very complex lesion. Although the qualitative and quantitative presence of these factors are now being exposed in burn and smoke injury, a detailed understanding of the effects and interrelationships of these mediators of inflammation in this injury is not yet available. For example, mRNA for both IL-1 and IL-8 are undetectable in the lung of normal sheep, but increase to high levels by 4 h after injury (FIG. 1).

Adherence of the neutrophil to the vascular endothelium precedes migration of the neutrophil into inflammatory sites. Adherence is mediated by at least three families of proteins expressed on the surface of the neutrophil or on the vascular endothelium. The β2 leukocyte integrins, lymphocyte function-associated antigen (LFA-1), macrophage antigen-1 (Mac-1), and p150,95, are adhesion glycoproteins expressed n the leukocyte surface (9-11). They are composed of heterogeneous α and common β subunits. ICAM-1, ICAM-2, and VCAM-1 are members of the immunoglobulin family of glycoproteins which are ligands for the leukocyte integrins (ICAM-1 AND ICAM-2) and for VLA-4, a β1 integrin (VCAM-1) (12-15). ICAM-1 is a ligand for CD11a/CD18 and also has affinity for CD11b/CD18. This ligand is expressed on endothelial cells, epithelial cells, and several other cell types and is inducible in vitro by inflammatory mediators such as TNF-α, IL-1β and IFN-γ (12,16).

The selecting, members of a family of lectin binding proteins, include L-selectin (also known as LECAM-1, the mouse lymphocyte homing receptor, MEL-14, and LAM-1, the human homologue of murine MEL-14), E-selectin (also known as the endothelial-leukocyte adhesion molecule-1, ELAM-1), and P-selectin (also known as PADGEM or GMP140) (14,17-19). L-selectin is located on the surface of the neutrophil, whereas E-selectin is present on endothelial cells. P-selectin occurs on both platelets and endothelial cells. The ligands for the selectins appear to be complex, mucin-like molecules (20). All selectins appear to bind to a minimum oligosaccharide moiety, sialyl Lewis x.

In contrast to the CD18 integrins, L-selectin is rapidly down-regulated within minutes of neutrophil activation (11,21,22). Treatment of neutrophils with antibody to L-selectin decrease adhesion of neutrophils to endothelium during both static conditions and those of high shear stress (23). Evidence that L-selectin is important for early neutrophil recognition of inflammatory sites includes findings that previously activated neutrophils, which have diminished L-selectin on their surfaces, do not localize to sites of inflammation (24). Pretreatment of neutrophils in vitro with at monoclonal antibody to L-selectin blocks neutrophil influx into inflamed peritoneum (24). Based on these and other experiments, a paradigm for neutrophil emigration from the microcirculation has been formulated (FIG. 2) (25). Rolling of a fraction of the blood neutrophils along the vascular endothelium is mediated by P-selectin. Immediate stimulation of the endothelium intensifies this effect. In addition, the ligand for L-selectin (which is not yet fully characterized) is upregulated on the endothelial cell surface (26). Within 4-6 h, E-selectin is maximally expressed and it is probable all the selectins participate in neutrophil capture from the circulation. Capture or “tethering” of the neutrophil, allows time for β2 integrins on the neutrophil surface to undergo conformational changes that increases their affinity for ICAM-1 (26). The participation β2 integrins is necessary for subsequent transmigration of the neutrophils through endothelial cell monolayers (27). In addition to these adherence events, PECAM (CD31), a member of the immunoglobulin supergene family clustered at tight junctions between endothelial cells, must interact with the neutrophil to allow passage (28). Recently, a third molecule, integrin associated protein (CD47) has also been implicated in the transendothelial migration of neutrophils(29). It is also likely that chemokines such as IL-8 and others direct the movement of neutrophils from the vasculature (30). While much evidence exists for this paradigm for the peripheral circulation, our and others experience with inflammatory lung conditions indicates that this paradigm requires alteration for application to the pulmonary circulation (31-38). Blocking of neutrophil emigration from the pulmonary circulation has so far not been possible in sheep with smoke and/or thermal injury. The purpose of the present study is to investigate the efficacy of a combined treatment with both anti-IL-8 and anti-L-selectin in blocking neutrophil emigration from the pulmonary vasculature.

Accordingly, it would be desirable to provide molecules that interact with the pro-inflammatory, inflammatory, and anti-inflammatory moieties that are induced in lung and burn lesions.

### BRIEF DESCRIPTION OF THE DRAWING FIGURES

FIG. 1 presents results of a reverse transcriptase polymerase chain reaction (RT-PCR) for the measurement of the mRNAs of IL-8 and IL-1β in lung.

FIG. 2 presents a schematic diagram of neutrophil emigration from the microcirculation.

FIG. 3 is a graph showing a comparison of net fluid balance required for resuscitation as required by animals undergoing severe burns in animals treated with anti-IL-8 or anti-L-selectin versus untreated animals.

FIG. 4 is a series of graphs examining lung lymph flow in animals treated with anti-IL-8 or anti-L-selectin versus untreated animals (FIGS. 4A and 4B) and a graph of levels of IL-8 in alveolar lavage.

FIG. 5 is a series of graphs examining plasma oncotic pressure, lung lymph oncotic pressure, and burned tissue lymph oncotic pressure in animals treated with anti-IL-8 or anti-L-selectin versus untreated animals.

FIG. 6 is a series of graphs examining cardiac index, mean arterial pressure, and pulmonary arterial pressure in animals treated with anti-IL-8 or anti-L-selectin versus untreated animals.

FIG. 7 is a series of graphs examining central venous pressure and left arterial pressure in animals treated with anti-IL-8 or anti-L-selectin versus untreated animals.

FIG. 8 is a series of graphs examining systemic vascular resistance index and pulmonary vascular resistance index in animals treated with anti-IL-8 or anti-L-selectin versus untreated animals.

FIG. 9 is a series of graphs examining burned tissue lymph flow and burned tissue lymph protein clearance in animals treated with anti-IL-8 or anti-L-selectin versus untreated animals.

FIG. 10 is a series of graphs examining burned tissue permeability index in animals treated with anti-IL-8 or anti-L-selectin versus untreated animals.

FIG. 11 is a series of graphs examining peripheral blood leukocytes (either white blood cells or neutrophils) in animals treated with anti-IL-8 or anti-L-selectin versus untreated animals.

FIG. 12 is a series of graphs examining lung permeability index in animals treated with anti-IL-8 or anti-L-selectin versus untreated animals.

### SUMMARY OF THE INVENTION

In accordance with a first aspect of the present invention, there is provided a method for the treatment of a mammal suffering from trauma associated with burns, comprising administering to the mammal at least one antibody that binds to a pro-inflammatory, inflammatory, or anti-inflammatory moiety that are induced in lung and burn lesions. In a preferred embodiment, the antibody comprises an anti-IL-8 antibody. In another preferred embodiment, the antibody comprises a monoclonal antibody. In another preferred embodiment, the antibody comprises a human antibody. In another preferred embodiment, the antibody comprises an anti-L-selectin antibody. In another preferred embodiment, the antibody comprises a monoclonal antibody.

In accordance with a second aspect of the present invention, there is provided a method for the treatment of a mammal suffering from trauma associated with burns comprising administering to the mammal at least one antibody that binds to interleukin-8. In a preferred embodiment, the antibody is a human antibody. In another preferred embodiment, the antibody is a monoclonal antibody.

In accordance with a third aspect of the present invention, there is provided a pharmaceutical composition for the treatment of a mammal suffering from trauma associated with burns or smoke inhalation comprising a monoclonal antibody that binds to interleukin-8 in a pharmaceutically acceptable carrier, filler, excipient, or diluent. In a preferred embodiment, the antibody is a human antibody. In another preferred embodiment, the antibody is a monoclonal antibody.

In accordance with a fourth aspect of the present invention, there is provided a pharmaceutical composition for the treatment of a mammal suffering from trauma associated with burns or smoke inhalation comprising a monoclonal antibody that binds to L-Selectin in a pharmaceutically acceptable carrier, filler, excipient, or diluent. In a preferred embodiment, the antibody is a human antibody. In another preferred embodiment, the antibody is a monoclonal antibody.

In accordance with a fifth aspect of the present invention, there is provided a method to reduce lung lymph flow in a mammal suffering from smoke inhalation injury or burn trauma comprising administering to the mammal at least one antibody that binds to a molecule selected from the group consisting of interleukin-8 and L-selectin. In a preferred embodiment, the antibody comprises a monoclonal antibody. In another preferred embodiment, the antibody comprises a human antibody.

### DETAILED DESCRIPTION OF THE PREFERRED EMBODIMENTS

In accordance with the present invention there are provided therapies for lung injuries following smoke inhalation and thermal injuries. In particular, the invention relates to the use of antibodies, and more particularly human antibodies, as therapeutics of such injuries. In preferred aspects of the invention, antibodies that bind to interleukin-8 (IL-8) and/or L-selectin, alone, or in combination with other antibodies that bind to other pro-inflammatory, inflammatory, and anti-inflammatory moieties that are induced in lung and burn lesions. In particularly preferred embodiments of the invention, human antibodies that bind to IL-8 are utilized and are demonstrated herein to possess therapeutic efficacy in in vivo models of the pro-inflammatory, inflammatory, and anti-inflammatory moieties that are induced in lung and burn lesions.

In accordance with the present invention, we utilized a sheep model (chronic ovine model) to examine lung injury associated with burn and/or smoke inhalation injury. In general, sheep were given a 40% surface burn while being subjected to smoke inhalation. Animals treated in accordance with the invention were compared with untreated control animals for a variety of criteria that are associated with lung injury. Neutrophil accumulation in lung parenchyma is characteristic of smoke inhalation. In the model, we that found these lung parenchymal alterations occurred more than 12 hours after smoke inhalation. Interleukin 8 (IL-8) is a potent chemokine. We have reported that the mRNA for lung IL-8 is upregulated following smoke inhalation. The objective for this study was to determine the effect of a monoclonal antibody (mAb) against IL-8 on lung fluid flux and the presence of IL-8 in lung lavage after smoke inhalation. In the model, sheep were surgically prepared with femoral arterial, venous, and lung lymph fistula. After a 5-7 day recovery period, the sheep received a 40% BSA 3rd° burn and 48 breaths of cotton smoke. The sheep were randomly assigned to 2 groups: A control group (n=7), and another was the treatment group (n=7) in which 1 mg/kg of IL-8 antibody (mAb) was administrated 2 hs before burn. After the injury, animals were mechanically ventilated and lung lymph flow was serially determined during the next 72 hour period of study. In additional sheep a single bronchial alveolar lavage was performed at 0 (n=1), 1 (n=2), 3 (n=3), 6 (n=2), and 12 h (n=3) post smoke.

In addition to examining the lung lymph flow and bronchial alveolar lavage, we also studied lung lymph protein clearance, net fluid balance, lung permeability, flank lymph flow, fluid resuscitation requirements, oxygen requirements, pulmonary toilet, epithelial damage, and numbers of neutrophils in the lung tissue. In accordance with the experiments, we demonstrated that both anti-IL-8 and anti-L-selectin antibodies decreased lung lymph flow (but not combination treatment with anti-IL-8 and anti-L-selectin), tended to decrease pulmonary microvascular permeability changes, and decreased flank lymph flows (though less than observed in the lung). In keeping with the decreased vascular leak, fluid resuscitation requirements were significantly less in the case of both anti-IL-8 and anti-L-selectin treatments.

In view of the data presented in connection with the present invention, it is expected that antibodies that bind to pro-inflammatory, inflammatory, and anti-inflammatory moieties that are induced in lung and burn lesions, and particularly antibodies to IL-8 or L-selectin are preferred.

Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g. Sambrook et al. Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g. free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutainine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to a EGF-r, under suitable binding conditions, (2) ability to EGF binding to its receptor, or (3) ability to inhibit EGF-r expressing cell growth in vitro or in vivo. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drus with properties analogous to those of the template peptide. These types of non-peptide compound are termed “peptide mimetics” or “peptidomimetics”. Fauchere, J Adv. Drug Res. 15:29 (1986); Veber and Freidinger TINS p.392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “biflnctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is≦1 μM, preferably≦100 nM and most preferably≦10 nM.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, 35S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

Human Antibodies and Humanization of Antibodies

Human antibodies avoid certain of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, it has been postulated that one can develop humanized antibodies or generate fully human antibodies through the introduction of human antibody function into a rodent so that the rodent would produce fully human antibodies.

Human Antibodies

The ability to clone and reconstruct megabase-sized human loci in YACs and to introduce them into the mouse germline provides a powerful approach to elucidating the functional components of very large or crudely mapped loci as well as generating useful models of human disease. Furthermore, the utilization of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (Mabs)—an important milestone towards fulfilling the promise of antibody therapy in human disease. Fully human antibodies are expected to minimize the immunogenic and allergic responses intrinsic to mouse or mouse-derivatized Mabs and thus to increase the efficacy and safety of the administered antibodies. The use of fully human antibodies can be expected to provide a substantial advantage in the treatment of chronic and recurring human diseases, such as inflammation, autoimmunity, and cancer, which require repeated antibody administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human Mabs with the desired specificity could be readily produced and selected.

This general strategy was demonstrated in connection with our generation of the first XenoMouse™ strains as published in 1994. See Green et al. Nature Genetics 7:13-21 (1994). The XenoMouse™ strains were engineered with yeast artificial chromosomes (YACs) containing 245 kb and 190 kb-sized germline configuration fragments of the human heavy chain locus and kappa light chain locus, respectively, which contained core variable and constant region sequences. Id. The human Ig containing YACs proved to be compatible with the mouse system for both rearrangement and expression of antibodies and were capable of substituting for the inactivated mouse Ig genes. This was demonstrated by their ability to induce B-cell development, to produce an adult-like human repertoire of fully human antibodies, and to generate antigen-specific human Mabs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively, to produce XenoMouse™ mice. See Mendez et al. Nature Genetics 15:146-156 (1997) and U.S. patent application Ser. No. 08/759,620, filed Dec. 3, 1996, the disclosures of which are hereby incorporated by reference.

Such approach is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996. See also Mendez et al. Nature Genetics 15:146-156 (1997) and Green and Jakobovits J. Exp. Med. 188:483-495 (1998). See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, and WO 98/24893, published Jun. 11, 1998. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, and 5,789,650 each to Lonberg and Kay, U.S. Pat. No. 5,591,669 to Krimpenfort and Berns, U.S. Pat. Nos. 5,612,205, 5,721,367, 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

The inventors of Surani et al., cited above and assigned to the Medical Research Counsel (the “MRC”), produced a transgenic mouse possessing an Ig locus through use of the minilocus approach. The inventors on the GenPharm International work, cited above, Lonberg and Kay, following the lead of the present inventors, proposed inactivation of the endogenous mouse Ig locus coupled with substantial duplication of the Surani et al. work.

An advantage of the minilocus approach is the rapidity with which constructs including portions of the Ig locus can be generated and introduced into animals. Commensurately, however, a significant disadvantage of the minilocus approach is that, in theory, insufficient diversity is introduced through the inclusion of small numbers of V, D, and J genes. Indeed, the published work appears to support this concern. B-cell development and antibody production of animals produced through use of the minilocus approach appear stunted. Therefore, research surrounding the present invention has consistently been directed towards the introduction of large portions of the Ig locus in order to achieve greater diversity and in an effort to reconstitute the immune repertoire of the animals.

Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against CTLA-4 in order to vitiate concerns and/or effects of HAMA or HACA response.

Humanization and Display Technologies

As was discussed above in connection with human antibody generation, there are advantages to producing antibodies with reduced immunogenicity. To a degree, this can be accomplished in connection with techniques of humanization and display techniques using appropriate libraries. It will be appreciated that murine antibodies or antibodies from other species can be humanized or primatized using techniques well known in the art. See e.g., Winter and Harris Immunol Today 14:43-46 (1993) and Wright et al. Crit, Reviews in Immunol. 12125-168 (1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al. P.N.A.S. 84:3439 (1987) and J.Immunol.139:3521 (1987)). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Antibody fragments, such as Fv, F(ab′).sub.2 and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segements to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. Mol Cell. Bio. 3:280 (1983)), Rous sarcoma virus LTR (Gorman et al. P.N.A.S. 79:6777 (1982)), and moloney murine leukemia virus LTR (Grosschedl et al. Cell 41:885 (1985)); native 1g promoters, etc.

Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Wright and Harris, supra., Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No. 5,733,743. If display technologies are utilized to produce antibodies that are not human, such antibodies can be humanized as described above.

Using these techniques, antibodies can be generated to CTLA-4 expressing cells, CTLA-4 itself, forms of CTLA-4, epitopes or peptides thereof, and expression libraries thereto (see e.g. U.S. Pat. No. 5,703,057) which can thereafter be screened as described above for the activities described above.

Additional Criteria for Antibody Therapeutics

As discussed herein, the function of the CTLA-4 antibody appears important to at least a portion of its mode of operation. By function, we mean, by way of example, the activity of the CTLA-4 antibody in operation and activity in the costimulatory pathway of CTLA-4. Accordingly, in certain respects, it may be desirable in connection with the generation of antibodies as therapeutic candidates against CTLA-4 that the antibodies be capable of fixing complement and participating in CDC. There are a number of isotypes of antibodies that are capable of the same, including, without limitation, the following: murine IgM, murine IgG2a, murine IgG2b, murine IgG3, human IgM, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. patent application Ser. No. 08/730,639, filed Oct. 11, 1996), among others.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

By way of example, the K2.2 antibody discussed herein is a human anti-IL-8 IgG2 antibody. Since such antibody appears to possess desired binding to the IL-8 molecule, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC.

Accordingly, as antibody candidates are generated that meet desired “structural” attributes as discussed above, they can generally be provided with at least certain of the desired “functional” attributes through isotype switching.

Therapeutic Administration and Formulations

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15^{th} ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration.

Preparation of Antibodies

Antibodies in accordance with the invention are preferably prepared through the utilization of a transgenic mouse that has a substantial portion of the human antibody producing genome inserted but that is rendered deficient in the production of endogenous, murine, antibodies. Such mice, then, are capable of producing human immunoglobulin molecules and antibodies and are deficient in the production of murine immunoglobulin molecules and antibodies. Technologies utilized for achieving the same are disclosed in the patents, applications, and references disclosed in the Background, herein. In particular, however, a preferred embodiment of transgenic production of mice and antibodies therefrom is disclosed in U.S. patent application Ser. No. 08/759,620, filed Dec. 3, 1996, the disclosure of which is hereby incorporated by reference. See also Mendez et al. Nature Genetics 15:146-156 (1997), the disclosure of which is hereby incorporated by reference.

Through use of such technology, we have produced fully human monoclonal antibodies to a variety of antigens. Essentially, we immunize XenoMouse™ lines of mice with an antigen of interest, recover lymphatic cells (such as B-cells) from the mice that express antibodies, fuse such recovered cells with a myeloid-type cell line to prepare immortal hybridoma cell lines, and such hybridoma cell lines are screened and selected to identify hybridoma cell lines that produce antibodies specific to the antigen of interest. We utilized these techniques in accordance with the present invention for the preparation of antibodies specific to IL-8, L-selectin, or other pro-inflammatory, inflammatory, and anti-inflammatory moieties that are induced in lung and burn lesions, such as PAF, as described above, or the other molecules discussed in the background.

In the present experiments, we utilized a fully human anti-IL-8 monoclonal antibody designated K2.2 that was generated through use of XenoMouse lines of mice. Such antibody was generated with and binds to human IL-8 and is cross reactive with sheep IL-8. Another antibody, designated D1.1, also generated from the XenoMouse lines of mice and capable of specifically binding human IL-8 was non-cross-reactive with sheep IL-8 and was essentially ineffective in the experiments described herein. Additional information related to the K2.2 and D1.1 anti-Il-8 antibodies is provided in U.S. patent application Ser. No. 08/759,620, filed Dec. 3, 1996, the disclosure of which is hereby incorporated by reference.

The anti-L-selectin antibody utilized in connection with this study is described in Spertini et al. J. Immunol. 147:942-949 (1991).

The hybridoma cell lines discussed herein are designated K2.2 and D1.1. Each of the antibodies produced by the aforementioned cell lines are fully human IgG2 heavy chains with human kappa light chains. In general, antibodies in accordance with the invention possess very high affinities, typically possessing Kd's of from about 10⁻⁹ through about 10⁻¹¹ M, when measured by either solid phase and solution phase.

As will be appreciated, antibodies in accordance with the present invention can be expressed in cell lines other than hybridoma cell lines. Sequences encoding particular antibodies can be used for transformation of a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introduction of heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels.

### EXAMPLES

The following examples, including the experiments conducted and results achieved are provided for illustrative purposes only and are not to be construed as limiting upon the present invention.

Methods

Sheep (n=25) were prepared surgically by placement of arterial, venous, left atrial and pulmonary arterial catheters. Pneumatic occluders were placed on the left pulmonary artery and the pulmonary veins. A catheter was positioned into the left pulmonary artery distal to the pneumatic occluder. The lung lymphatics were cannulated and the nonlung components of the drainage of the lung lymphatic were eliminated by cutting the distal portion of the caudal mediastinal lymph node and cauterizing the surface of the diaphragm.

The animals were given 5 to 7 days to recover prior to study. Before study the sheep were connected to pressure monitoring devices and maintenance fluids (2 ml/kg/hr lactated Ringer's solution) were begun. A urinary retention catheter was positioned and baseline data were collected 24 h later. If these variables were within normal limits, blood samples were obtained for the various laboratory studies, the sheep anesthetized and an inhalation injury induced using a modified bee smoker and smoke generated from burning cotton toweling materials. Concurrently, a 40% full-thickness surface burn was induced over the animals side.

The sheep were then placed on ventilators and the anesthetic discontinued. Positive end-expiratory pressure was set to 5 cm H₂O, tidal volume at 15 ml/kg, and frequency and FiO₂ were adjusted to maintain PaO₂>80 mmHg and PaCO₂<40 mmHg. The animals were divided into three groups (n=12): Group I: Anti-L-selectin+anti-IL-8 (both 1 mg/kg); Group II: anti-IL-8 (1 mg/kg); Group III: Anti-L-selectin+anti-IL-8 (both 1 mg/kg); and Group IV: Saline sham treatment. The treatment were given two hours before smoke insufflation and thermal injury to insure equilibrium of the antibody in the body fluids.

The animals were studied for 72 h. ELISA assays for IL-8 and FACS analysis for L-selectin indicated that the concentration of antibodies was sufficient throughout the 72 h study period. The following physiological variables were recorded at 4-h intervals: mean aortic, pulmonary left and right atrial pressures, and cardiac output. Bronchial blood flows were determined by the colored microsphere technique at baseline, 6 h, and then at 12-h intervals.

Systemic and pulmonary vascular resistances were calculated and indexed to body surface area using standard formulas. Arterial and mixed venous blood were assayed for PO₂, PCO₂, hemoglobin saturation, and base excess. Pneumatic cuffs on the left pulmonary artery and veins were inflated and the bronchial venous blood flow into the pulmonary circulation was measures. Lung lymph flow was measured with a graduated test tube and stopwatch.

Samples of mixed venous, bronchial venous, and arterial blood, and lung lymph were mixed with various chilled anticoagulants, centrifuged at SoC, and stored at 80° C. These samples were used for analysis of IL-1 and IL-8, total protein, oncotic pressure, conjugated dienes, and soluble selecting. Samples of whole blood were used for the determination of cell counts and hematocrit. At necropsy, samples of blood were drawn for analysis of wet-dry ratios and myeloperoxidase. Samples of lung, ileum, spleen and pancreas were obtained and immediately placed into liquid nitrogen for analysis of mRNA for IL-1 and IL-8, conjugated dienes, myeloperoxidase, and wet/dry weight. Samples for histology were placed into formalin solution. Inmunohistochemistry techniques are being developed with polyclonal antibodies made specific for E- and P-selectins to determine tissue levels of ICAM-1 and E- and P-selectins to determine tissue levels of ICAM-1 and E- and P-selectin in the future.

Discussion

Unlike the lung microvascular permeability index (FIG. 12), the IL-8 treatment group exhibits no difference in peripheral tissue permeability from the control animals (FIG. 10). Although statistical significance was not reached, the anti-L-selectin treatment group does show a trend towards less permeability than the control.

Surprisingly, peripheral blood leukocyte numbers were reduced in some of the treatment animals (FIG. 11). Most of the decrease in peripheral blood leukocytes could be account for by decreased numbers of neutrophils. Although there was a trend towards increased total leukocyte and neutrophil numbers in the peripheral blood of the anti-Il-8 treated animals, these differences were not statistically significant.

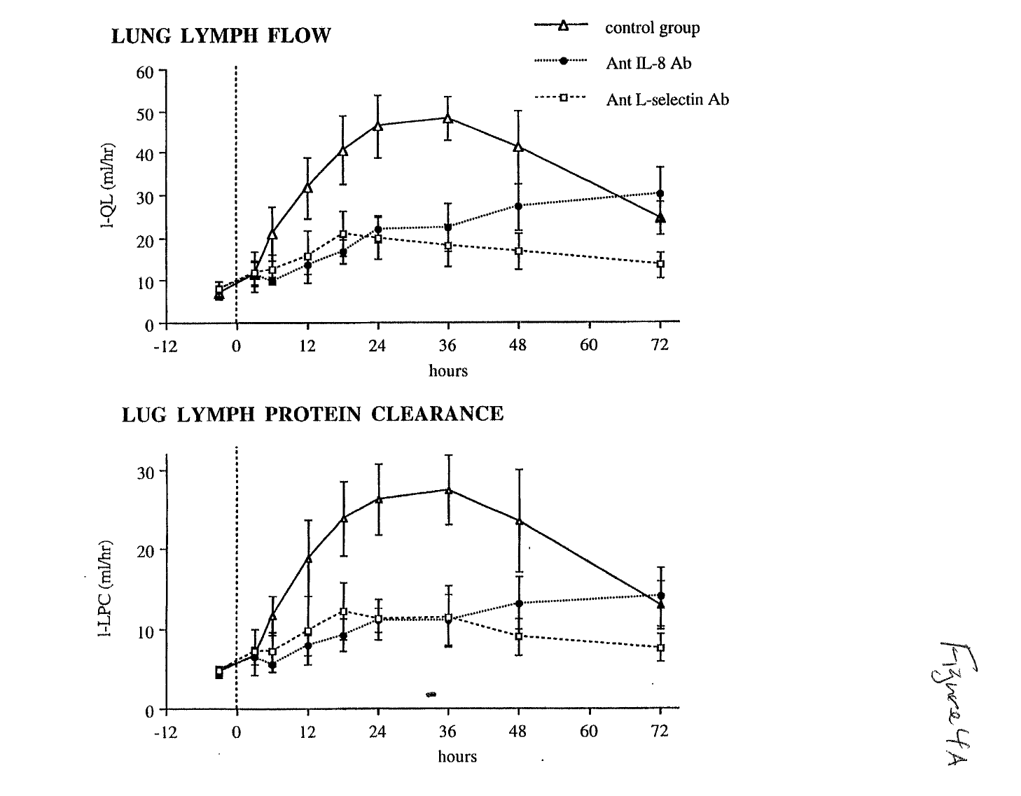

Net Fluid Balance

Where animals undergo severe burns, they require immediate fluid resuscitation. Fluid therapy is adjusted to maintain mean arterial pressure (MAP). In FIG. 3, we demonstrate that anti-IL-8 significantly reduces the net fluid balance requirement in the tested animals.

Lung Lymph Flow and Lung Lymph Protein Clearance

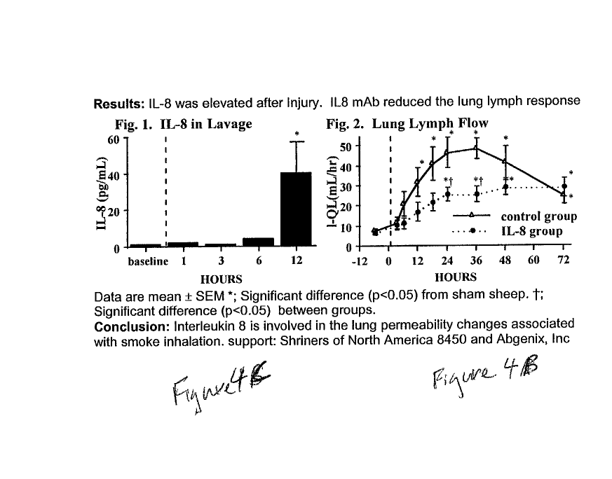

In FIG. 4A and 4B, we demonstrate the decreased lung lymph flow for both anti-IL-8 and anti-L-selectin. This data is consistent with less interstitial fluid in the lungs which is consistent with the net fluid balance data (note that the amount of fluid in the lungs cannot account for the improved fluid balance—there has to be less fluid leak in the peripheral tissues as well). Things become more complicated when we try to understand the mechanisms by which the decrease in interstitial fluid occurs. In FIG. 4C, the levels of IL-8 in alveolar lavage is shown.

Other Pressures and Flows

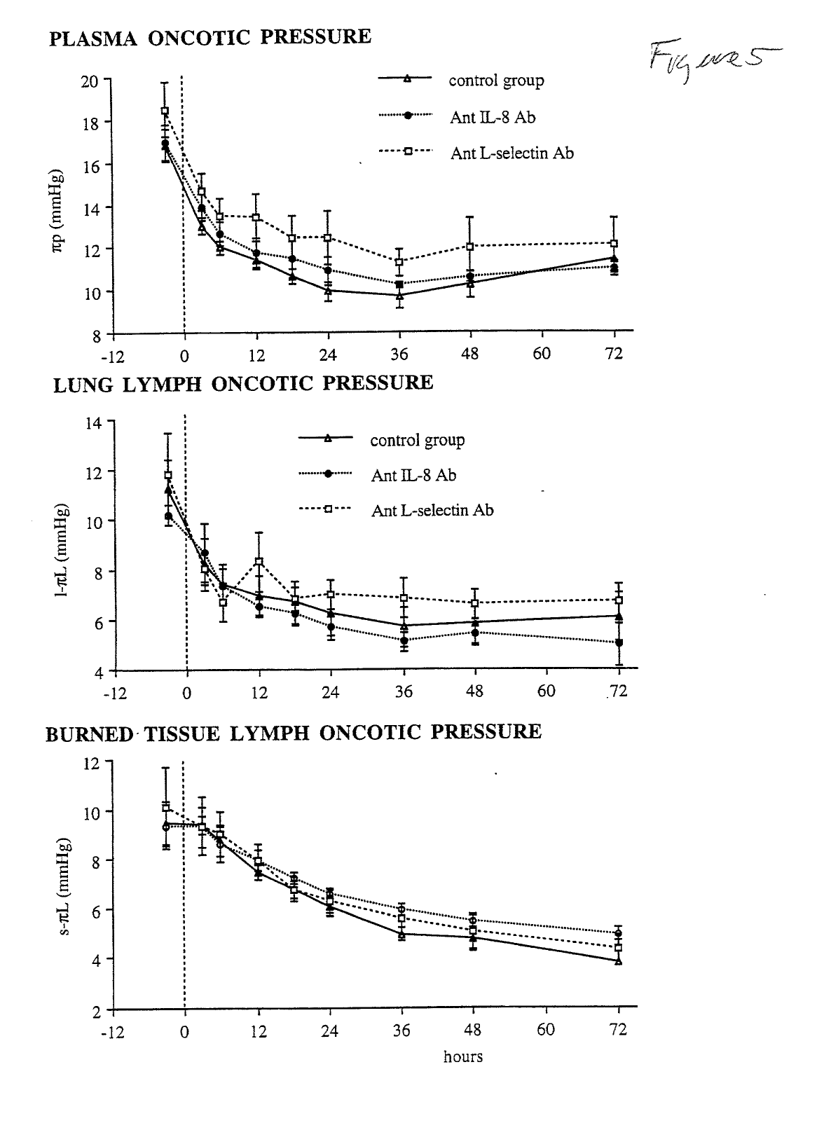

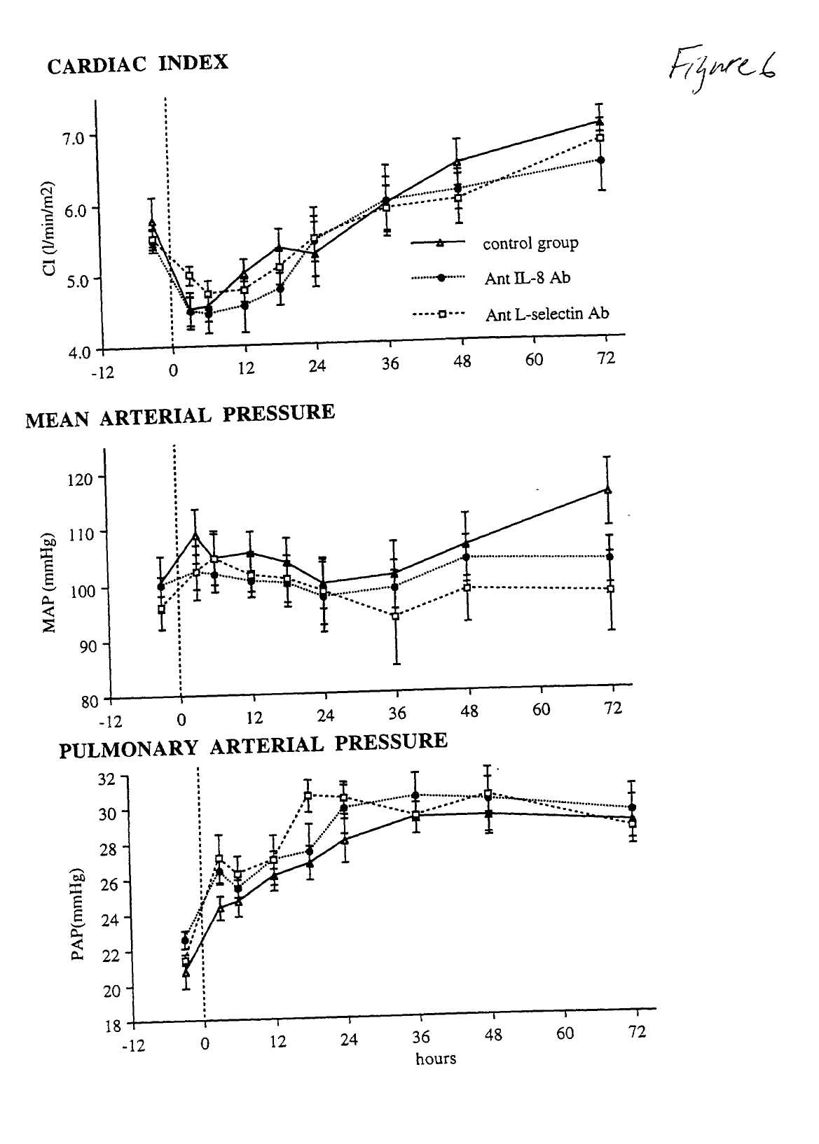

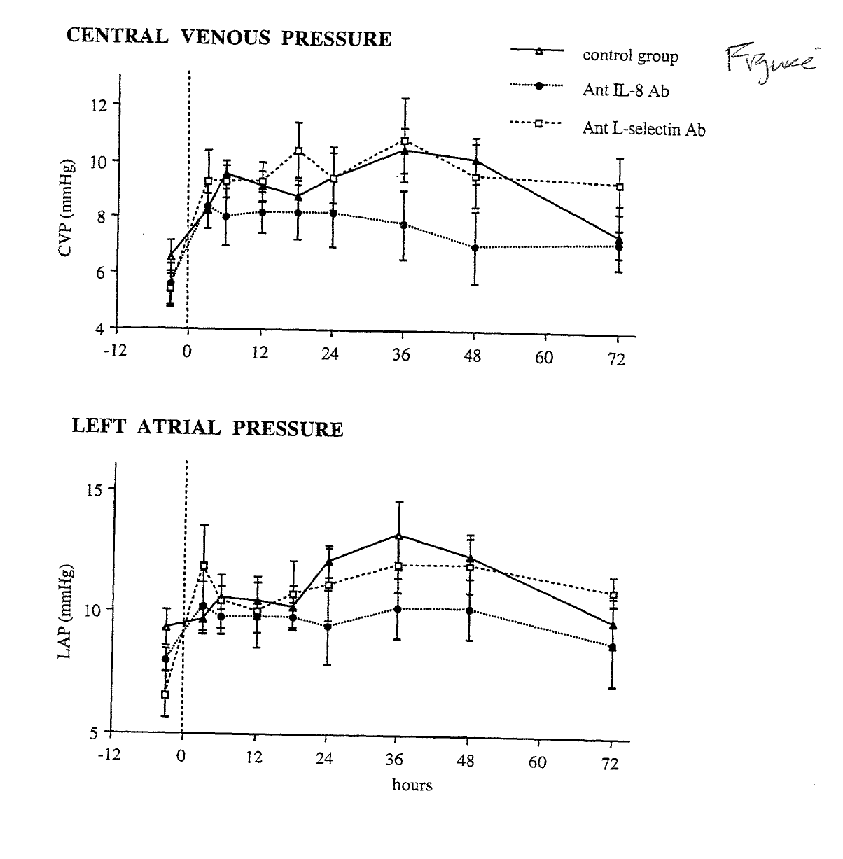

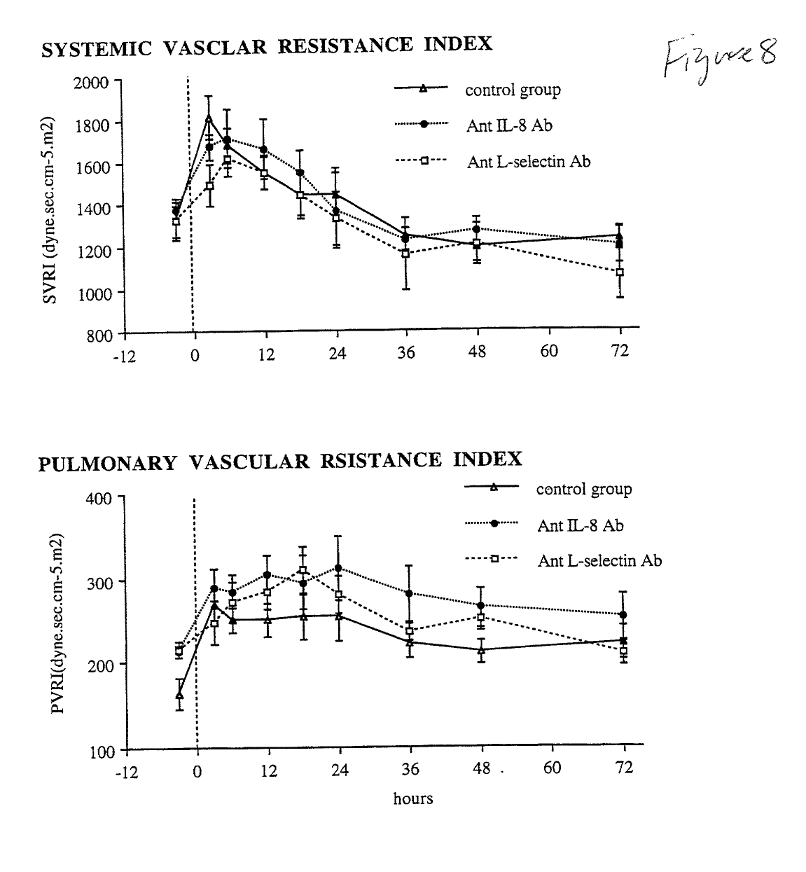

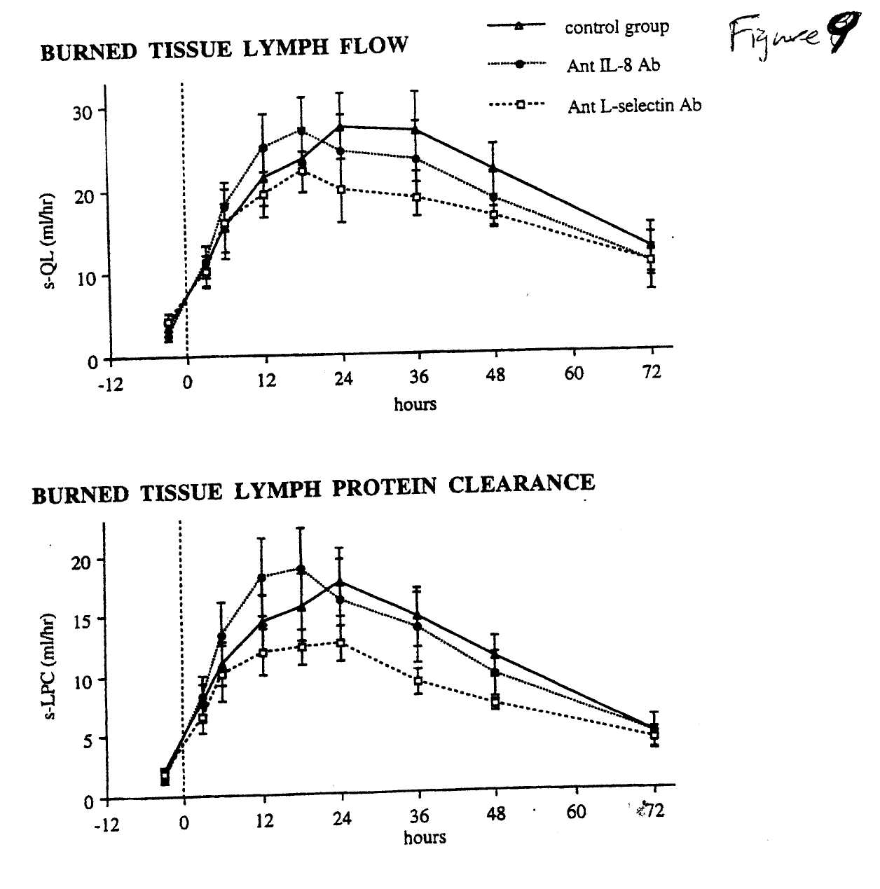

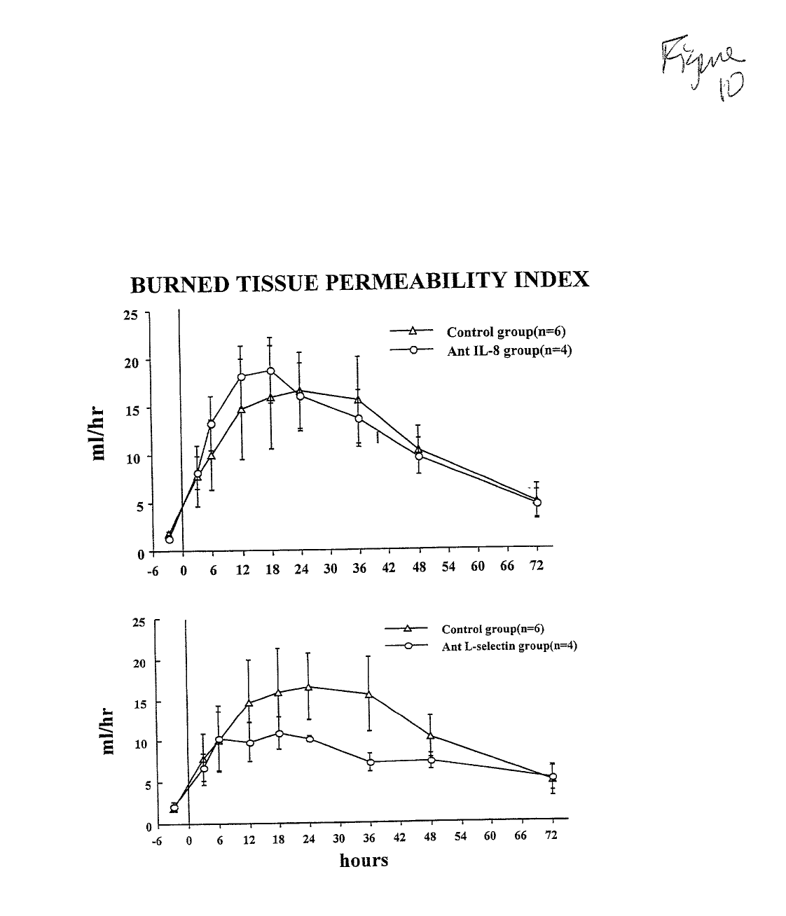

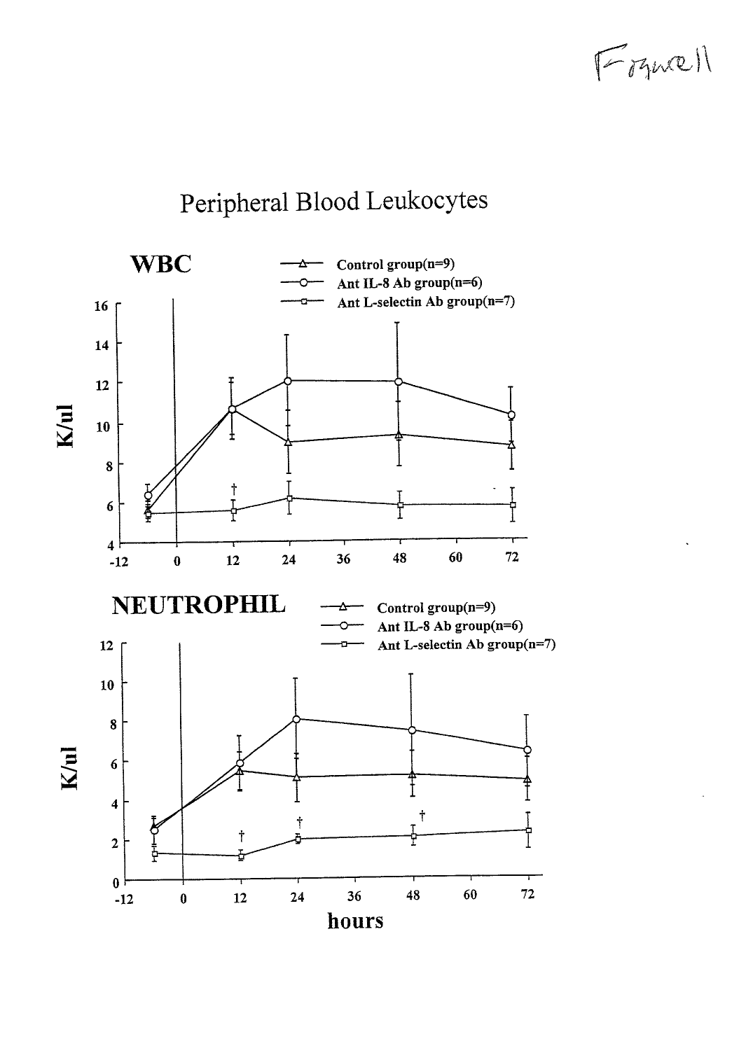

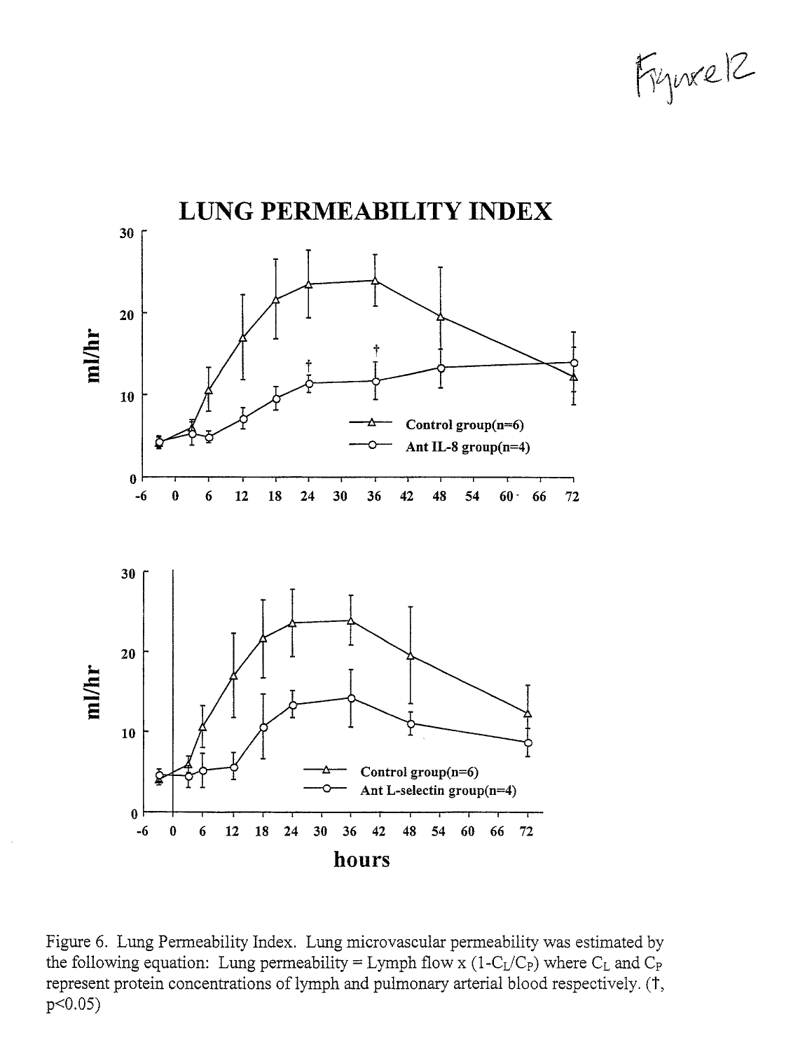

In FIGS. 5-8, a variety of peripheral pressures and flows were investigated. In FIG. 5, plasma oncotic pressures, lung lymph oncotic pressures and burned flank lymph oncotic pressures are illustrated for the control and two treatment groups. There is not enough difference in these data to account for the decrease in interstitial fluid that is clearly occurring with the treatment groups. It could be postulated that more microvascular beds were open in the two treatment groups with a concommitant drop in microvascular pressures. However, in FIG. 6, the cardiac index is no different among any of the groups which is not consistent with this notion. Neither is mean arterial pressure or pulmonary arterial pressure different. In FIG. 7, central venous pressure is lower in the anti-IL-8 group as is left atrial pressure, but the differences are probably not significant. Also, in FIG. 8, systemic vascular resistance and pulmonary vascular resistance does not appear to be different among the three groups. Finally in FIG. 10, burned tissue lymph flow is unchanged. However, no differences among the groups is perhaps not surprising in view of the likelihood that the damage to these tissues is irreversible.

Basically, explanations of the interstitial fluid shifts in these animals based on changes in oncotic pressures or hydrostatic pressures do not appear likely. We will be able to make some estimations of microvascular bed pressures based on the data we have. However, we may again find no significant differences. The more probable explanation for this behavior has to do with the properties of the microvascular vessels namely their permeability to water (hydraulic conductivity, Lp, in the Starling equation, Jᵥ/S=Lₚ[Pᵢ−σ(π_{c}−π_{I})]). Unfortunately, to calculate this quantity, the reflection coefficient is required and this parameter has not yet been measured in our animals because it requires transient occlusion of the pulmonary veins which alters the histology of the lung. Nevertheless, some estimates of this parameter may be made in other ways and we are presently pursuing these estimates.

In thinking about this surprising possibility of blocking of increased hydraulic permeability by anti-IL-8, the unexpected effects of anti-IL-8 and anti-L-selectin given together, and our suspicion that neutrophil activation might be involved, we considered that platelet activating factor as well as IL-8 might be a candidate mediator in the smoke and burn lesion. Indedd, a paper (Harris and Granger, Am J. Physiol. 170:H127-H133 (1996)) bears directly on this issue. The authors found that infused PAF is capable of increasing Lₚ in rat mesentery. Furthermore, it is known that this response is neutrophil-mediated. Significantly, they found that infusion of Sle^{x} blunted this response as did a combination of SOD and catalase. Their conclusion was that selectins and reactive oxygen intermediates (presumably contributed by neutrophils) were important in this action of PAF. By analogy: Activated neutrophils roll along both post-capillary venules and capillaries and produce PAF that partially mediates the lesion seen in this smoke and burn model. IL-8 produced by the neutrophil may act on the endothelial cell or in an autocrine fashion on the neutrophil to amplify PAF production. Therefore, either anti-IL-8 or anti-L-selectin has some effect on this process. However, when antibodies bind to L-selectin on the neutrophil surface and perhaps to IL-8 bound either to A or B type CXC chemokine receptors, the neutrophil may be further stimulated to make more reactive oxygen intermediates and more PAF under this “double” stimulus. Since L-selectin may only partially interfere with neutrophil rolling, this scenario might result in apparent abrogation of the effect of either antibody used alone.

In accordance with the experiments, we demonstrated that both anti-IL-8 and anti-L-selectin antibodies decreased lung lymph flow (but not combination treatment with anti-IL-8 and anti-L-selectin), tended to decrease pulmonary microvascular permeability changes, and decreased flank lymph flows (though less than observed in the lung). In keeping with the decreased vascular leak, fluid resuscitation requirements were significantly less in the case of both anti-IL-8 and anti-L-selectin treatments. In contrast to the effects of anti-L-selectin and anti-IL-8 on vascular permeability and resuscitation fluid requirements, there was no discernible effect on oxygen requirements or pulmonary toilet. Histological examination of injured lung tissue showed similar amounts of epithelial damage among the treatment groups and the controls. Consistent with this finding, the numbers of neutrophils in the lung tissue (scored blindly) were not different among the various groups. This finding suggests that neither anti-IL-8, anti-L-selectin, or a combination of the two antibodies are able to block neutrophil migration in the lung. In addition, recent evidence suggests that thermal injuries may increase apoptosis of neutrophils. Since these tests were conducted at 72 hours post trauma, it is possible that many such cells may have undergone apoptotic events and disintegrated by such time point. Thus, it is possible that neutrophil numbers may have been different earlier in the life of the lesion and the histology may be underestimating the numbers of neutrophils in the burned animals.

The effects on vascular permeability may indicate that both anti-IL-8 and anti-L-selectin decrease endothelial cell injury. It is even possible that by blocking both IL-8 and L-selectin, these neutrophils were less activated at the time they emigrated in the lung parenchyma. This may even have allowed a more severe injury on arrival at the epithelial cells. This is a possible explanation for the certain of the decreased efficacy of the combined anti-IL-8 and anti-L-selectin therapies.

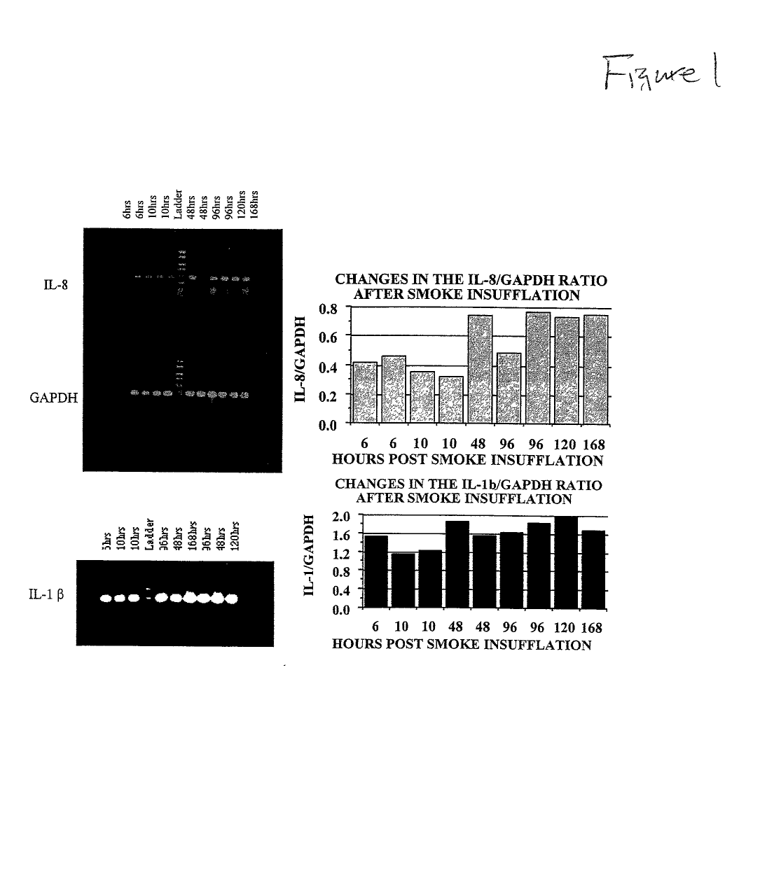

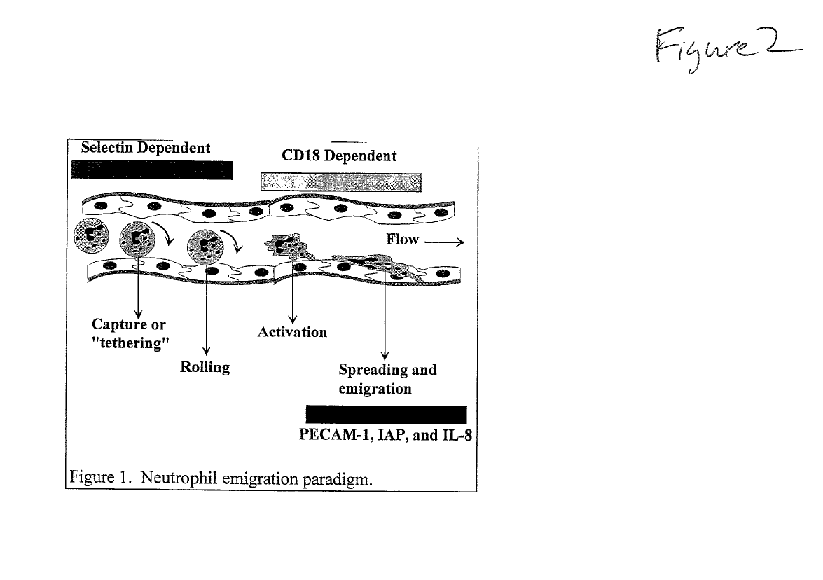

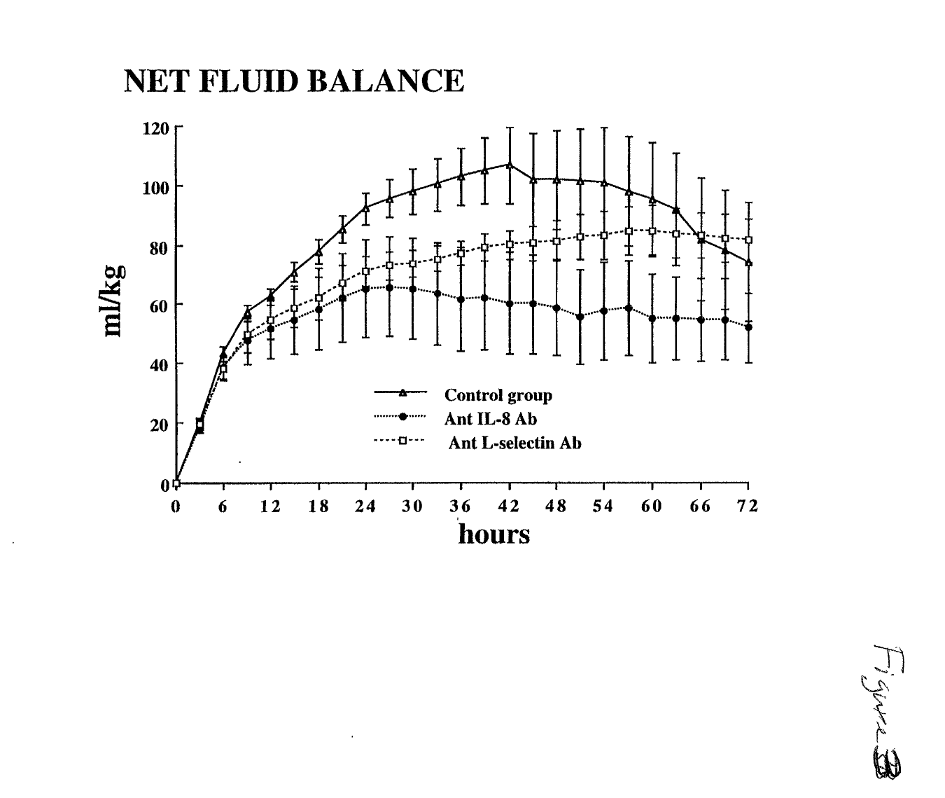

The question of why the combined treatment with anti-IL-8 and anti-L-selectin are not effective at reducing neutrophil migration into the lung is puzzling. In the case of IL-8, we have already shown that IL-8 mRNA is present with 4 h after the lesion is produced (FIG. 1). In FIG. 1, the data collected from sheep that had been insufflated with smoke from burning cotton toweling material and then sacrificed at the time intervals indicated above. Messenger RNA levels for IL-1α were similar to that found for IL-1β (data not shown). We have been unable to demonstrate expression of the mRNAs of these cytokines in lung tissue of uninjured animals. These data are compared to the mRNA of glyceraldehyde-3-phospate dehydrogenase (GAPDH, an internal control) a noninducible enzyme. After smoke insufflation, the mRNAs for the cytokines were continuously expressed for at least 168 h. There is a similarity between the time course of changes in transvascular fluid and changes in IL-1 and IL-8 mRNA levels. The data shown is a representation experiment in which each time point was collected from a single sheep. The mRNA remains at high levels throughout the next 72 h. However, IL-8 is not the only CXC cytokine produced in the lung. GRO-α was shown to be present at high levels in some experimental lung lesions (39,40). Similarly, ENA-78 and GCP-2 may also play some role. As for anti-L-selectin, it may not be sufficient since both E- and P-selectins are capable of acting in a process of neutrophil “capture,” which may in certain cases be a more efficient process in the lung. If this were to prove true, blockade of all selectin activity could be indicated in order to completely block neutrophil entry.

The potential role of complement should also be considered in this process. It is possible that the rapid epithelial damage produced by the toxic chemicals in the smoke may expose neoantigens that react with circulating antibody that in turn can activate the classical complement pathway. Perhaps a more likely possibility is that the damaged epithelial cells present a suitable surface for alternative pathway activation. Either pathway could result in the production of C5 and subsequently C5a that could act as a powerful neutrophil chemoattractant. We have previously demonstrated that neutrophils may move independently of adhesive molecules and towards chemoattractants under some conditions (41). Presumably, this motion occurs through hydraulic and shape change mechanisms. It is possible that this mechanism serves for neutrophil emigration into the lung in this thermal and smoke injury model.

Finally, the effect of anti-L-selectin on peripheral blood neutrophil counts is interesting. It has been previously postulated that L-selectin functions as an adherence molecule regulating neutrophil egress from the bone marrow (42). However, other previous data put this view in some doubt (43). It is likely that another mechanism accounts for the lowered peripheral blood neutrophil numbers.

Additional results and discussions related to the use of the anti-L-selectin antibody is provided in Annex I, the disclosure of which is hereby incorporated by reference.

### INCORPORATION BY REFERENCE

All references cited herein, including patents, patent applications, papers, text books, and the like, and the references cited therein, to the extent that they are not already, are hereby incorporated herein by reference in their entirety. In addition, the following references are also incorporated by reference herein in their entirety, including the references cited in such references:

1. Hemdon, D N; Barrow, R E; Linares, H A; Rutan, R L; Prien, T; Traber, L D; Traber, D L (1988) Inhalation injury in burned patients; effects and treatments. Burns. Incl. Therm. Inj. 14, 349-356.

2. Demling, R H; LaLonde, C (1990): Moderate smoke inhalation produces decreased oxygen delivery, increased oxygen demands, and systemic but not lung parenchymal lipid peroxidation. Surgery 108,544.

3. Hemdon, D N; Ziegler, S T (1993): Bacterial translocation after thermal injury. [Review]. Crit. Care Med. 21, S50-S54.

4. Knox, J; Youn, Y K; Demling, R (1992): Burn edema is accentuated by a moderate smoke inhalation injury in sheep. Surgery 112, 908-917.

5. Demling, R; Picard, L; Campbell, C; La Londe, C (1993): Relationship of burn-induced lung lipid peroxidation on the degree of injury after smoke inhalation and a body burn. Crit. Care Med. 21, 1935-1943.

6. Ono, I; Gunji, H; Zhang, J Z; Maruyama, K; Kaneko, F (1995): A study of cytokines in burn blister fluid related to wound healing. Burns. 21,352-355.

7. Wu, J Z; Ogle, C K; Fischer, J E; Warden, G D; Ogle, J D (1995): The mRNA expression and in vitro production of cytokines and other proteins by hepatocytes and Kupffer cells following thermal injury. Shock. 3,268-273.

8. O'Sullivan, S T; Lederer, J A; Horgan, A F; Chin, D H; Mannick, J A; Rodrick, M L (1995): Major injury leads to predominance of the T helper-2 lymphocyte phenotype and diminished interleukin-12 production associated with decreased resistance to infection. Ann. Surg. 222, 482-490.

9. Anderson, D C; Schmalstieg, F C; Kohl, S; Hughes, B J; Tosi, M F; Buffone, G J; Dickey, W D; Abramosn, J S; Boxer, L A; Brinkley, B R; Smith, C W (1984): Abnormalities of polymorphonuclear leukocyte function associated with a heritable deficiency of a high molecular weight surface glycoprotein (GP138): common relationship to diminished cell adherence. J. Clin. Invest. 74, 546-561.

10. Springer, T A; Thompson, W S; Miller, L J; Schmalstieg, F C; Anderson, D C (1984): Inherited deficiency of the Mac-1, LFA-1, p150,95 glycoprotein family and its molecular basis. J. Exp. Med. 160, 1901-1918.

11. Kishimoto, T K; Jutila, M A; Berg, E L; Butcher, E C (1989): Neutrophil Mac-1 and MEL-14 adhesion proteins inversely regulated by chemotactic factors. Science. 245(4923, 15 Sep), 1238-1241.

12. Pohlman, T H; Stanness, K A; Beatty, P G; Ochs, H D; Harlan, J M (1986): an endothelial cell service factor(s) induced in vitro by lipopolysaccharide, interleukin 1, and tumor necrosis factor-alpha increases neutrophil adherence by a CDw18-dependent mechanism. J. Immunol. 136, 4548-4553.

13. Smith, C W; Rothlein, R; Hughes, B J; Mariscalco, M M; Rudloff, H E; Schmalstieg, F C; Anderson, D C (1988): Recognition of an endothelial determinant for CD18-dependent human neutrophil adherence and transendothelial migration. J. Clin. Invest. 82, 1746-1756.

14. Haskard, D O; Lee, T H; (1992): The role of leukocyte-endothelial interactions in the accumulation of leukocytes in allergic inflammation. [Review]. Am. Rev. Respir. Dis. 145(2, Pt 2, Feb), S10-S13.

15. Rothlein, R; Dustin, M L; Marlin, S D; Springer, T A (1986): an intercellular adhesion molecule (ICAM-1) distinct from LFA-1. J. Immunol. 137, 1-5.

16. Rothlein, R; Czajkowski, M; O'Neill, M M; Marlin, S D; Mainolfi, E; Merluzzi, V J (1988): Induction of intercellular adhesion molecule 1 on primary and continuous cell lines by pro-inflammatory cytokines. Regulation by pharmacologic agents and neutralizing antibodies. J. Immunol. 141(5, 1 Sep) 1665-1669.

17. Kishimoto, T K; Jutila, M A; Butcher, E C (1990): Identification of a human peripheral lymph node homing receptor: a rapidly down-regulated adhesion molecule. Proc. Natl. Acad. Sci. (USA) 87(6, Mar), 2244-2248.

18. Yednock, T A; Butcher, E C; Stoolman, L M; Rosen, S D (1987): Receptors involved in lymphocyte homing: relationship between a carbohydrate-binding receptor and the MEL-14 antigen. J. Cell Biol. 104(3, Mar), 725-731.

19. Lewinsohn, D M; Bargatze, R F; Butcher, E C (1987): Leukocyte-endothelial cell recognition: evidence of a common molecular mechanism shared by neutrophils, lymphocytes, and other leukocytes. J. Immunol. 138(12, 15 Jun), 4313-4321.

20. Tamatani, T; Suematsu, M; Tezuka, K; Hanzawa, N; Tsuji, T; Ishimura, Y; Kannagi, R; Toyoshima, S; Homma, M (1995): Recognition of consensus CHO structure in ligands for selectins by novel antibody against sialyl Lewis X. Am. J. Physiol. 269, H1282-H1287.

21. Jutila, M A; Rott, L; Berg, E L; Butcher, E C (1989): Function and regulation of the neutrophil MEL-14 antigen in vivo: comparison with LFA-1 and MAC-1. J. Immunol. 143(10, 15 Nov), 3318-3324.

22. Griffin, J D; Spertini, O; Ernst, T J; Belvin, M P; Levine, H B; Kanakura, Y; Tedder, T F (1990): Granulocyte-macrophage colony-stimulating factor and other cytokines regulate surface expression of the leukocyte adhesion molecule-1 on human neutrophils, monocytes, and their precursors. J. Immunol. 145(2, 15 Jul), 576-584.

23. Smith, C W; Kishimoto, T K; Abbassi, O; Hughes, B; Rothlein, R; McIntire, L V; Butcher, E; Anderson, D C; Abbassi, O (1991): Chemotactic factors regulate lectin adhesion molecule 1 (LECAM-1)-dependent neutrophil adhesion to cytokine-stimulated endothelial cell in vitro [published erratum appears in J Clin Invest 1991 May; 87(5):1873]. J. Clin. Invest. 87(2, Feb), 609-618.

24. Jutila, M A; Rott, L; Berg, E L; Butcher, E C (1989): Function and regulation of the neutrophil MEL-14 antigen in vovo comparison with LFA-1 and Mac-1. J. Immunol. 143, 3318-3324.

25. Springer, T A (1994); Traffic signals for lymphocyte recirculation and leukocyte emigration: the multistep paradigm. [Review]. Cell. 76(2, 28 Jan), 301-314.

26. Tedder, T F; Steeber, D A; Chen, A; Engel, P (1995): The selectins: vascular adhesion molecules. [Review]. FASEB 9, 866-873.

27. Smith, C W; Rothlein, R; Hughes, B J; Mariscalco, M M; Rudloff, H E; Schmalstieg, F C; Anderson, D C (1988): Recognition of an endothelial determinant for CD 18-dependent human neutrophil adherence and transendothelial migration. J. Clin. Invest. 82(5, Nov), 1746-1756.

28. Bogen, S; Pak, J; Garifallou, M; Deng, X; Muller, W A (1994): Monoclonal antibody to murine PECAM-1 (CD31) blocks acute inflammation in vivo. J. Exp. Med. 179, 1059-1064.

29. Cooper, D; Lindberg, F P; Gamble, J R; Brown, E J; Vadas, M A (1995): Transendothelial migration of neutrophils involves integrin-associated protein (CD47). Proc. Natl. Acad. Sci. (USA) 92, 3978-3982.

30. Smith, W B; Gamble, J R; Clark-Lewis, I; Vadas, M A (1993): Chemotactic desensitization of neutrophils demonstrates interleukin-8 (IL-8)-dependent and IL-8-independent mechanisms of transmigration through cytokine-activated endothelium.

31. Doerschuk, C M; Winn, R K; Coxson, H O; Harlan, J M (1990): CD18-dependent and independent mechanisms of neutrophil emigration in the pulmonary and systemic microcirculation of rabbits. J. Immunol. 144 (6, 15 Mar), 2327-2333.

32. Bullard, D C; Qin, L; Lorenzo, I; Quinlin, W M; Doyle, N A; Bosse, R; Vestweber, D; Doerschuk, C M; Beaudet, A L (1995): P-selectin/ICAM-1 double mutant mice: acute emigration of neutrophils into the peritoneum is completely absent but is normal into pulmonary alveoli [see comments]. J. Clin. Invest. 95(4, Apr), 1782-1788.

33. Burns, A R; Doerschuk, C M (1994): Quantitation of L-selectin and CD18 expression on rabbit neutrophils during CD18-independent and CD18-dependent emigration in the lung. J. Immunol. 153(7, Oct), 3177-3188.

34. Doerschuk, C M; Markos, J; Coxson, H O; English, D; Hogg, J C (1994): Quantitation of neutrophil migration in acute bacterial pneumonia in rabbits. J. Appl. Physiol. 77(6, Dec), 2593-2599.

35. Keeney, S E; Mathews, M J; Haque, A K; Rudloff, H E; Schmalstieg, F C (1994): Oxygen-induced lung injury in the guinea pig proceeds through CD18-independent mechanisms. Am. J. Respir. & Crit. Care Med. 149(2, Pt. 1, Feb), 311-319.

36. Guha, S C; Hemdon, D N; Evans, M J; Schmalstieg, F C; Isago, T; Traber, L D; Linares, H A; Traber, D L (1993): Is the CD18 adhesion complex of polymorphonuclear leukocytes involved in smoke-induced lung damage? A morphometric study. J. Burn Care and Rehabil. 14(5, Sep-Oct), 503-511.

37. Mulligan, M S; Polley, M J; Bayer, R J; Nunn, M F; Paulson, J C; Ward, P A (1992): Neutrophil-dependent acute lung injury. Requirement for P-selectin (GMP-140). J. Clin. Invest. 90(4, Oct), 1600-1607.

38. Mulligan, M S; Till, G O; Smith, C W; Anderson, D C; Miyasaka, M; Tamatani, T; Todd, R F, 3^{rd}; Issekutz, T B; Ward, P A (1994): Role of leukocyte adhesion molecules in lung and dermal vascular injury after thermal trauma of skin. Am. J. Pathol. 144(5, May), 1008-1015.

39. Mulder, K; Colditz, I G (1993): Migratory responses of ovine neutrophils to inflammatory mediators in vitro and in vivo. Journal. of. Leukocyte. Biology. 53(3, Mar), 273-278.

40. Ahuja, S K; Murphy, P M (1996): The CXC chemokines growth-regulated oncogene (GRO) alpha, GRObeta, GROgamma, neutrophil-activating peptide-2, and epithelial cell-derived neutrophil-activating peptide-78 are potent agonists for the type B, but not the type A, human interleukin-8 receptor. Journal of Biological Chemistry. 271(34, 23 Aug), 20545-20550.

41. Schmalstieg, F C; Rudloff, H E; Hillman, G R; Anderson, D C (1986): Two-dimensional and three-dimensional movement of polymorphonuclear leukocytes: two fundamentally different mechanisms of locomotion. Journal. of. Leukocyte. Biology. 40, 677-691.

42. van Eeden, S F; Miyagashima, R; Haley, L; Hogg, J C (1997): A possible role for L-selectin in the release of polymorphonuclear leukocytes from bone marrow. American. Journal. of. Physiology. 272(4, Pt 2, Apr), H1717-H1724.

43. Jagels, M A; Chambers, J D; Arfors, K E; Hugli, T E (1995): C5a- and tumor necrosis factor-alpha-induced leukocytosis occurs independently of beta 2 integrins and L-selectin: differential effects on neutrophil adhesion molecule expression in vivo. Blood. 85(10, 15 May), 2900-2909.

### Equivalents

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What we claim is:

1. A method for the treatment of a mammal suffering from trauma associated with burns, comprising administering to the mammal at least one antibody that binds to a pro-inflammatory, inflammatory, or anti-inflammatory moiety that are induced in lung and burn lesions.

2. The method of claim 1, wherein the antibody comprises an anti-IL-8 antibody.

3. The method of claim 2, wherein the antibody comprises a monoclonal antibody.

4. The method of claim 2, wherein the antibody comprises a human antibody.

5. The method of claim 3, wherein the antibody comprises a human antibody.

6. The method of claim 1, wherein the antibody comprises an anti-L-selectin antibody.

7. The method of claim 6, wherein the antibody comprises a monoclonal antibody.

8. A method for the treatment of a mammal suffering from trauma associated with burns, comprising administering to the mammal at least one antibody that interleukin-8.

9. The method of claim 8, wherein the antibody is a human antibody.

10. The method of claim 9, wherein the antibody is a monoclonal antibody.

11. A pharmaceutical composition for the treatment of a mammal suffering from trauma associated with burns or smoke inhalation comprising a monoclonal antibody that binds to interleukin-8 in a pharmaceutically acceptable carrier, filler, excipient, or diluent.

12. The pharmaceutical composition of claim 11, wherein the antibody is a human antibody.

13. The pharmaceutical composition of claim 12, wherein the antibody is a monoclonal antibody.

14. A pharmaceutical composition for the treatment of a mammal suffering from trauma associated with burns or smoke inhalation comprising a monoclonal antibody that binds to L-Selectin in a pharmaceutically acceptable carrier, filler, excipient, or diluent.

15. The pharmaceutical composition of claim 14, wherein the antibody is a human antibody.

16. The pharmaceutical composition of claim 15, wherein the antibody is a monoclonal antibody.

17. A method to reduce lung lymph flow in a mammal suffering from smoke inhalation injury or burn trauma comprising administering to the mammal at least one antibody that binds to a molecule selected from the group consisting of interleukin-8 and L-selectin.

18. The method of claim 1, wherein the antibody comprises a monoclonal antibody.

19. The method of claim 2, wherein the antibody comprises a human antibody.
